(19) United States
(12) Patent Application Publication
(10) Pub. No.: US 2023/0265059 A1
(43) Pub. Date: Aug. 24, 2023

(54) SOLID STATE FORMS OF 6-CARBOXY-2-(3, 5-DICHLOROPHENYL)-BENZOXAZOLE OF FORMULA-I AND PHARMACEUTICALLY ACCEPTABLE SALTS THEREOF

(71) Applicant: NURAY CHEMICALS PRIVATE LIMITED, Thiruvallur (IN)

(72) Inventors: Venkatragavan RAMASAMY, Thimvallur, Tamil Nadu (IN); Balaiah ERUGU, Thimvallur, Tamil Nadu (IN); Ajay Madhukar SINGAVARAPU, Thimvallur, Tamil Nadu (IN); Vinayagam NALLAPPAN, Thimvallur, Tamil Nadu (IN); Saravanan SIVAPRAGASAM, Thimvallur, Tamil Nadu (IN); Vijayakumar KURAPATI, Thimvallur, Tamil Nadu (IN); Silambarasan RAMAN, Thimvallur, Tamil Nadu (IN)

(21) Appl. No.: 18/004,064

(22) PCT Filed: Jul. 3, 2021
(86) PCT No.: PCT/IN2021/050649

§ 371 Date: Jan. 2, 2023

(30) Foreign Application Priority Data
Jul. 4, 2020 (IN) 202041028537
Jan. 25, 2021 (IN) 202141003402

(51) Int. Cl. C07D 263/57; C07C 231/12; C07C 215/10

(52) U.S. Cl. CPC C07D 263/57; C07C 231/12; C07C 215/10; C07B 2200/13

(57) ABSTRACT

The present disclosure relates to novel solid forms of 6-carboxy-2-(3,5-dichlorophenyl)-benzoxazole of Formula (I) and it pharmaceutically acceptable salts thereof.

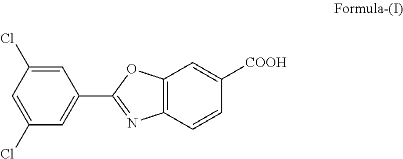 [SMILES: O=C(O)c1ccc2nc(-c3cc(Cl)cc(Cl)c3)oc2c1]

### RELATED APPLICATIONS

This patent application claims the benefit of priority of Indian patent application bearing number 202041028537 dated 4 Jul. 2020 which is incorporated herein by reference in its entirety.

### FIELD OF THE INVENTION

The present disclosure relates to novel solid forms of 6-carboxy-2-(3,5-dichlorophenyl)-benzoxazole of Formula-I and its pharmaceutically acceptable salts thereof.

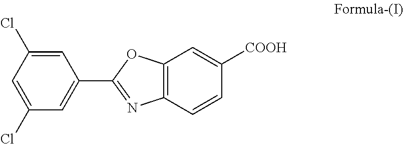 [SMILES: O=C(O)c1ccc2nc(-c3cc(Cl)cc(Cl)c3)oc2c1]

### BACKGROUND OF THE INVENTION

The 6-carboxy-2-(3,5-dichlorophenyl)-benzoxazole of formula (I) and 6-carboxy-2-(3,5-dichlorophenyl)-benzoxazole 1-deoxy-1-methylamino-D-glucitol or meglumine salt of Formula-(IA) are indicated for the treatment of transthyretin-related hereditary amyloidosis. Transthyretin is a homotetrameric protein present in serum and cerebral spinal fluid and the main function is the transport of L-thyroxine and the holo-retinol-binding-protein.

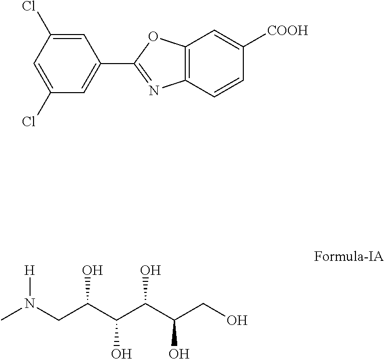 [SMILES: O=C(O)c1ccc2nc(-c3cc(Cl)cc(Cl)c3)oc2c1 ; CNC[C@H](O)[C@@H](O)[C@H](O)[C@H](O)CO]

U.S. Pat. No. 7,214,695 B2 described a process for the preparation of 6-carboxy-2-(3,5-dichlorophenyl)-benzoxazole as given below scheme

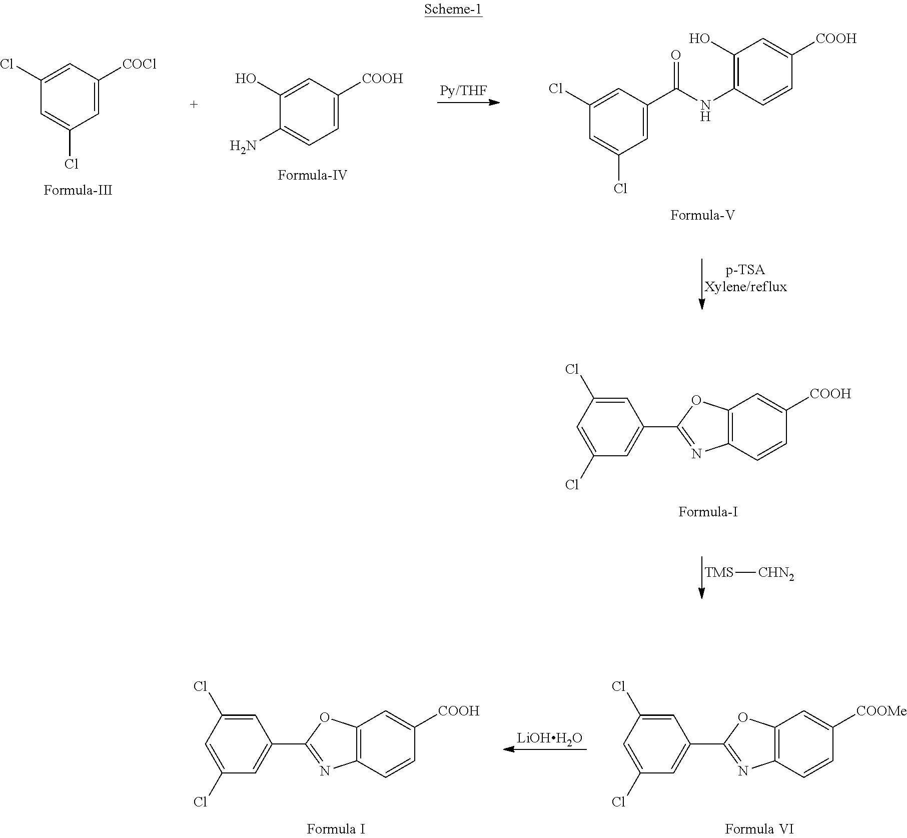

The main drawback of this patent is use of hazardous and highly toxic reagent such as pyridine and trimethylsilyldiazomethane for the preparation of compound of formula-I and the methylating agent trimethylsilyldiazomethane is very expensive; hence this process is not safe and cost effective. Moreover the final compound is isolated by using thin layer chromatography which is not viable in commercial scale process.

U.S. Pat. No. 8,168,663 described the pharmaceutically acceptable salt of 6-carboxy-2-(3,5-dichlorophenyl)-benzoxazole of formula (I) more specifically meglumine salt of 6-carboxy-2-(3,5-dichlorophenyl)-benzoxazole of Formula-IA.

U.S. Pat. No. 9,249,112 B2 patent described a crystalline form, liquid crystal form, amorphous form of 6-carboxy-2-(3,5-dichlorophenyl)-benzoxazole meglumine of formula (IA) and process for preparation thereof.

U.S. Pat. No. 9,770,441 B1 described crystalline Form 1, Form 2, Form 4 and Form 6 of 6-carboxy-2-(3,5-dichlorophenyl)-benzoxazole of compound of formula (I) and is also disclosed process for the preparation of compound of formula I as given below scheme.

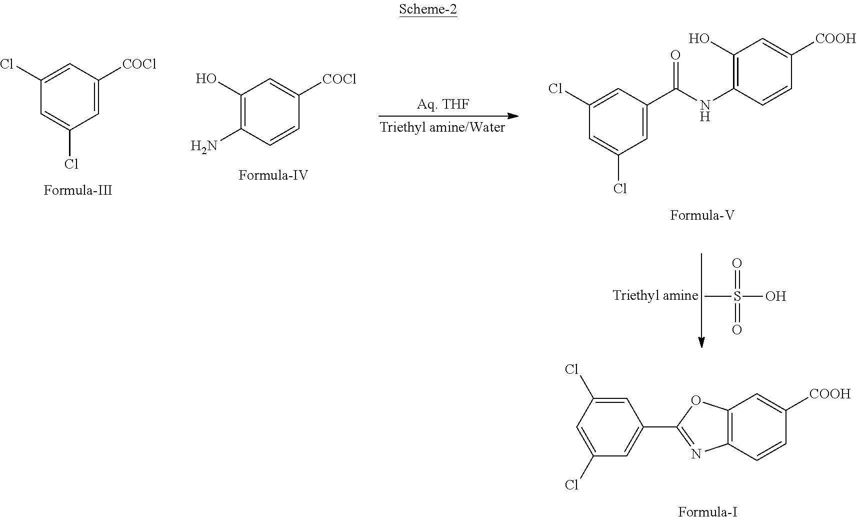

Even though this patent provides good yield and quality this patent process involves tedious workup procedures, the compounds obtained in the reaction mass are coloured and it required multiple purifications and isolation of the compound from the reaction mass is very difficult. Hence this process is only viable for small scale production and it is not useful for commercial process

U.S. Pat. No. 10,577,334 B2described the Form-E of Tafamidis meglumine and process for its preparation.

WO2019175263A 1 described the various polymorphs of Tafamidis meglumine and Tafamidis free acid.

WO2020207753A1 described the process for the preparation of Tafamidis meglumine without isolation of Tafamidis free acid by cyclising ester of compound of Formula (VII) and hydrolysing ester with sodium or potassium hydroxide and followed by extraction of Tafamidis in tetrahydrofuran and addition of meglumine in tetrahydrofuran solution. In this process, the ester compound of Formula (VII) is prepared by esterifying 4-amino-3-hydroxy-benzoic acid of compound of Formula IV followed by coupling of ester with 3,5-dichloro-benzoyl chloride of Formula-III as given below scheme.

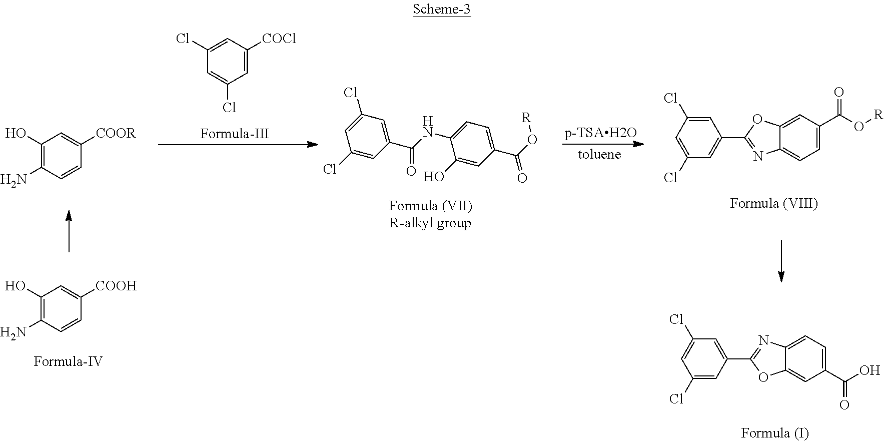

This patent publication is not disclosed process for the preparation of the ester compound of Formula (IV). In conventional methods, the ester compound has been prepared by using acid or acid chlorides such as sulfuric acid or hydrochloric acid or acetyl chloride. If the hydrochloric acid is used for esterification, the reaction mass needs be cooled to 0° C. and the reaction will take atleast 48 hours to complete for 5 g scale followed by workup procedures. This process is tedious, requires high conventional cost which makes this process not viable in commercial scale. The sulfuric acid process requires reflux temperature which is not user friendly for commercial scale due to its own drawback of handling and hazardous nature.

Hence there is a need for a simple, industrially feasible and commercially viable process for the preparation of 6-carboxy-2-(3,5-dichlorophenyl)-benzoxazole of compound of formula (I) and its pharmaceutically acceptable salts. The present inventors surprisingly found a simple process for the preparation of 6-carboxy-2-(3,5-dichlorophenyl)-benzoxazole of compound of formula (I) and its pharmaceutically acceptable salts.

### Advantages of the Invention

The present disclosure is to provide a simple and cost effective process for the preparation of 6-carboxy-2-(3,5-dichlorophenyl)-benzoxazole of Formula (I).

The present disclosure is to provide a process for the preparation of 6-carboxy -2-(3,5-dichlorophenyl)-benzoxazole which is suitable for large scale preparation and economically viable.

The present disclosure is to provide stable crystalline forms of 6-carboxy-2-(3,5-dichlorophenyl)-benzoxazole of formula (I) and its pharmaceutically acceptable salts.

### SUMMARY OF THE INVENTION

In first embodiment, the present invention provides a novel crystalline form of 6-carboxy-2-(3,5-dichlorophenyl)-benzoxazole of formula (I) (herein after designated as “Form L”) and its process for the preparation thereof.

In second embodiment, the present invention provides a novel crystalline forms of 6-carboxy-2-(3,5-dichlorophenyl)-benzoxazole of formula (I) (herein after designated as “Form P”) and its process for the preparation thereof.

In third embodiment, the present invention provides a novel process for the preparation of Form-4 of 6-carboxy-2-(3,5-dichlorophenyl)-benzoxazole of formula (I).

In fourth embodiment, the present invention provides, novel crystalline salts of 6-carboxy-2-(3,5-dichlorophenyl)-benzoxazole of formula (I) and its process for preparation thereof.

In fifth embodiment, the present invention provides a novel crystalline Hydrate form of 6-carboxy-2-(3,5-dichlorophenyl)-benzoxazole meglumine of formula (IA) and its process for preparation thereof.

In sixth embodiment, the present disclosure provides a process for the preparation 6-carboxy-2-(3,5-dichlorophenyl)-benzoxazole of formula (I) or its pharmaceutically acceptable salts thereof which comprising steps of:
i. esterifying 4-(3,5-dichloro-benzoylamino)-3-hydroxy-benzoic acid of formula

(V) to obtain 4-(3,5-dichloro-benzoylamino)-3-hydroxy-benzoic acid alkyl ester of formula (VII);
ii. cyclizing 4-(3,5-dichloro-benzoylamino)-3-hydroxy-benzoic acid alkyl ester to obtain 2-(3,5-dichlorophenyl)-benzoxazole-6-carboxylic acid alkyl ester of Formula (VIII);
iii. hydrolysing 2-(3,5-dichlorophenyl)-benzoxazole-6-carboxylic acid alkyl ester of Formula (VIII) to obtain 6-carboxy-2-(3,5-dichlorophenyl)-benzoxazole of formula (I);
iv. optionally converting 6-carboxy-2-(3,5-dichlorophenyl)-benzoxazole of formula (I) to its Base salt of formula (II);
v. hydrolysing 6-carboxy-2-(3,5-dichlorophenyl)-benzoxazole Base salt of formula (II) to obtain 6-carboxy-2-(3,5-dichlorophenyl)-benzoxazole formula (I); and
vi. optionally converting 6-carboxy-2-(3,5-dichlorophenyl)-benzoxazole of formula (I) obtained from step-iii) or step-v) to its pharmaceutically acceptable salt.
   The present disclosure is represented by following scheme

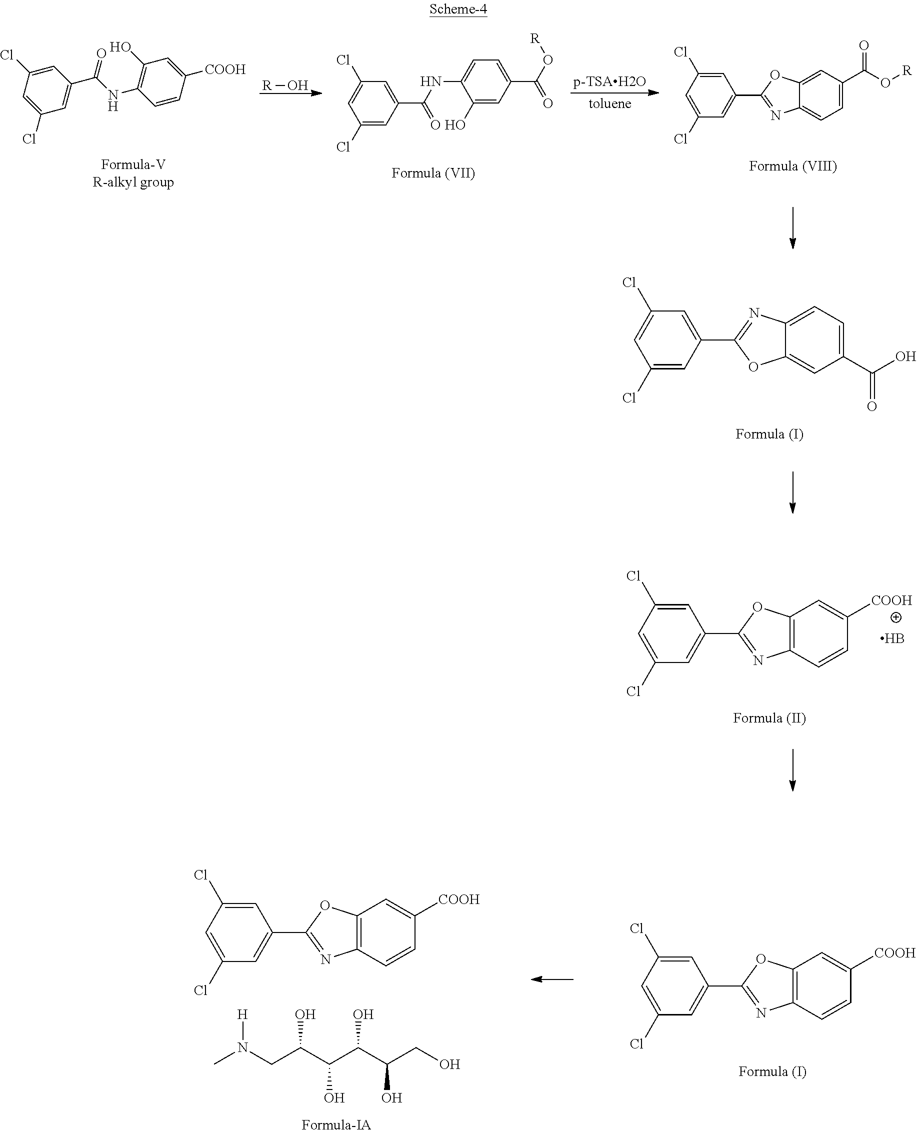

In seventh embodiment, the present invention provides a process for the preparation of novel salts of 6-carboxy-2-(3,5-dichlorophenyl)-benzoxazole of formula (II).

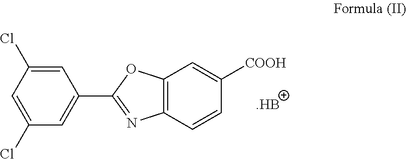

wherein the “HBE)” is basic salts which comprising steps of:
a) providing 6-carboxy-2-(3,5-dichlorophenyl)-benzoxazole of formula (I) or its ester of compound of formula (VIII) in a solvent;
b) optionally hydrolysing ester of compound of formula (III);
c) adding base to the reaction mass;
d) optionally heating the reaction mass; and
e) isolating novel salt of 6-carboxy-2-(3,5-dichlorophenyl)-benzoxazole of formula (II).

### BRIEF DESCRIPTION OF DRAWING

FIG. 1 shows DSC of Hydrate Form of 6-carboxy-2-(3,5-dichlorophenyl) -benzoxazole meglumine.

FIG. 2 shows TGA of Hydrate Form of 6-carboxy-2-(3,5-dichlorophenyl) -benzoxazole meglumine.

### DETAILED DESCRIPTION OF THE INVENTION

The following paragraphs detail various embodiments of the invention. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent important aspects of the invention that may be taken in isolation and also combined with other important aspects of the invention described elsewhere within this specification as a whole, and including the examples and figures. The skilled person will appreciate that the invention extends to such combinations of features and that these have not been recited in detail here in the interests of brevity.

Definitions of some of the terms used herein are detailed below.

The use of the terms “a” and “an” and “the” and similar references in the context of describing the crystalline forms described herein (especially in the context of the following claims) and process thereof are to be construed to cover both the singular and the plural, unless otherwise indicated herein or clearly contradicted by context.

The term “about” as used herein embodies standard error associated with a physico-chemical observable. As used herein, the term “about” means a slight variation of the value specified, for example, within 10% of the value specified. A stated amount for a compositional ingredient that is not preceded by the term “about” does not mean that there is no variance for the stated term, as one of ordinary skill would understand that there may be the possibility of a degree of variability generally associated with experimental error.

As used herein, the term “activation” mean that the activation of compound of Formula (V) by activating group to form acid chloride, ester, amide and anhydride.

In an embodiment, the crystalline Form-L of 6-carboxy-2-(3,5-dichlorophenyl)-benzoxazole of formula (I) has a PXRD pattern comprising peaks at 6.4, 13.0, 22.0 and 27.2±0.2 °2θ. In another embodiment of the present disclosure, the novel polymorph of ]

\6-carboxy-2-(3,5-dichlorophenyl)-benzoxazole of formula (I) has PXRD pattern at 9.52, 10.9, 16.7, 19.7, 23.8 and 44.6±2θ. Still one more aspect of the present disclosure, the novel polymorph of 6-carboxy-2-(3,5-dichlorophenyl) -benzoxazole of formula (I) was characterised by PXRD pattern as shown by following table-1.

TABLE 1
PXRD Results of Form L of 6-carboxy-2-(3,5-dichlorophenyl)-
benzoxazole

| Pos. [º2θ] | d-spacing [Å] | Rel. Int. [%] |
|---|---|---|
| 3.9228 | 22.52489 | 5.55 |
| 5.0996 | 17.32932 | 5.34 |
| 6.4486 | 13.70668 | 34.97 |
| 9.5253 | 9.28529 | 11.88 |
| 10.9872 | 8.05289 | 20.37 |
| 13.0526 | 6.78288 | 35.16 |
| 15.4366 | 5.74028 | 18.62 |
| 15.9234 | 5.56590 | 32.13 |
| 16.7245 | 5.30106 | 35.09 |
| 17.3683 | 5.10596 | 23.67 |
| 17.4981 | 5.06840 | 23.20 |
| 19.3888 | 4.57821 | 35.76 |
| 19.7631 | 4.49233 | 43.50 |
| 20.4732 | 4.33809 | 14.99 |
| 22.0955 | 4.02312 | 29.59 |
| 23.8658 | 3.72855 | 52.66 |
| 25.4523 | 3.49963 | 46.27 |
| 26.1153 | 3.41226 | 48.96 |
| 27.2512 | 3.27255 | 93.52 |
| 31.7261 | 2.82044 | 7.85 |
| 35.0451 | 2.56056 | 4.84 |
| 36.4582 | 2.46450 | 3.77 |
| 38.3827 | 2.34524 | 6.65 |
| 41.9197 | 2.15518 | 5.44 |
| 44.6232 | 2.03069 | 100.00 |

Still one more embodiment, crystalline Form-P of 6-carboxy-2-(3,5-dichlorophenyl)-benzoxazole of formula (I) has a PXRD pattern comprising peaks at 6.6, 12.5, and 22.8±2θ. In another embodiment, the crystalline Form-P of 6-carboxy -2-(3,5-dichlorophenyl)-benzoxazole of formula (I) has PXRD pattern at 7.5, 10.7, 16.5, 18.4, 19.6, 25.0 and 44.6±0.2 °2θ. Still one more aspect of the present disclosure the crystalline Form-P of 6-carboxy-2-(3,5-dichlorophenyl)-benzoxazole of formula (I) was characterised by PXRD pattern as shown in following table-2

TABLE 2
PXRD Results of Form P of 6-carboxy-2-(3,5-dichlorophenyl)-
benzoxazole

| Pos. [º2θ] | d-spacing [Å] | Rel. Int. [%] |
|---|---|---|
| 6.5659 | 13.46209 | 100.00 |
| 7.5095 | 11.77265 | 10.66 |
| 10.6670 | 8.29389 | 7.34 |
| 12.4172 | 7.12852 | 33.31 |
| 12.9963 | 6.81214 | 5.19 |
| 14.9993 | 5.90667 | 11.89 |
| 16.4120 | 5.40127 | 27.15 |
| 18.3530 | 4.83418 | 20.50 |
| 19.5625 | 4.53794 | 9.09 |
| 20.4545 | 4.34201 | 2.21 |
| 21.8184 | 4.07358 | 3.50 |
| 22.7194 | 3.91402 | 41.51 |
| 23.1804 | 3.83722 | 6.64 |
| 24.2973 | 3.66330 | 6.84 |
| 24.9200 | 3.57316 | 38.75 |
| 25.5447 | 3.48717 | 6.46 |
| 25.8336 | 3.44883 | 8.31 |
| 26.2983 | 3.38893 | 6.23 |
| 26.9689 | 3.30617 | 18.90 |
| 27.6818 | 3.22263 | 11.36 |
| 29.2195 | 3.05644 | 2.55 |
| 29.7809 | 3.00008 | 4.42 |
| 30.8809 | 2.89568 | 1.25 |
| 31.6525 | 2.82683 | 1.44 |
| 32.0398 | 2.79354 | 3.17 |
| 33.1012 | 2.70635 | 2.03 |
| 34.5768 | 2.59416 | 2.60 |
| 38.3726 | 2.34583 | 1.79 |
| 39.5319 | 2.27967 | 1.74 |
| 44.6177 | 2.03092 | 12.81 |

In an embodiment, the present disclosure provides a process for the preparation of Form-4 comprising
a) providing compound of formula (V);
b) adding acid to the reaction mass;
c) heating the reaction mass to about 80 to about 130° C. optionally in presence of solvent; and
d) adding suitable solvent to precipitate form-4 of compound of formula (I).
   wherein the acid is selected from methane-sulfonic acid, ethanesulfonic acid, phenylmethane sulfonic acid, camphor-10-sulfonic acid, naphthalene-1-sulfonic acid, naphthalene-2-sulfonic acid, naphthalene-1,5-disulfonic acid, sulfuric acid, fumaric acid, camphor sulfonic acid, phosphoric acid and polyphosphoric acid or a combination thereof and suitable solvent in step d) is selected from methanol, ethanol, isopropanol, acetone, methyl ethyl ketone, methyl isobutyl ketone ethyl acetate, acetonitrile, diisopropylether, methyl tertiarybutyl ether and dioxane, toluene, hexane, cyclohexane and heptane.

The present inventors surprisingly found that the cyclising compound of formula (V) using methanesulfonic acid in absence of solvent results Form-4. The present inventors also observed that stirring the 6-carboxy-2-(3,5-dichlorophenyl) -benzoxazole with fumaric acid in presence of solvent or solvent combination at temperature in the range of 50 to 90° C. resulting the Form-4 of 6-carboxy-2-(3,5-dichlorophenyl)-benzoxazole. The PXRD of wet material and the final compound obtained from the reaction is matching with Form-4 as disclosed in U.S. Pat. No. 9,770,441.

The crystalline Form-4 or new forms such as Form-L and Form-P obtained from present disclosure can be converted in to other solid forms of 6-carboxy-2-(3,5-dichlorophenyl)-benzoxazole of formula (I) and the solid forms of 6-carboxy-2-(3,5-dichlorophenyl)-benzoxazole meglumine salt of formula (IA).

In an embodiment, the esterification of 4-(3,5-dichloro-benzoylamino)-3-hydroxy-benzoic acid of formula (V) is carried out by activating acid to obtain acid halide followed by esterification by using respective alcohols. The preferred acid activation reaction product is acid chloride and it is prepared by the reaction of acid of Formula (V) with thionyl chloride or oxalyl chloride. The alkanol used in step (i) is selected from the group consisting of methanol, ethanol, propanol, isopropanol, butanol, isobutanol, t-butanol, 1-pentanol and 2-pentanol preferably methanol.

In another embodiment, the cyclization reaction is carried out in presence of acid selected from the group consisting of methane-sulfonic acid, ethanesulfonic acid, phenylmethanesulfonic acid, camphor-10-sulfonic acid, naphthalene-1-sulfonic acid, naphthalene-2-sulfonic acid, naphthalene-1,5-disulfonic acid and sulfuric acid, fumaric acid, phosphoric acid and polyphosphoric acid or a combination thereof.

In another embodiment, suitable solvent in step d) is selected from methanol, ethanol, isopropanol, butanol, acetone, methyl ethyl ketone, methyl isobutyl ketone ethyl acetate, acetonitrile, diisopropylether, methyl tertiarybutyl ether, dioxane, toluene, anisole, hexane, cyclohexane and heptane or combination thereof

In another embodiment, the ester hydrolysis is carried out by using base selected from group consisting of sodium hydroxide, potassium hydroxide, lithium hydroxide, barium hydroxide, caesium hydroxide, sodium carbonate, potassium carbonate, barium carbonate, caesium carbonate, sodium bicarbonate, potassium bicarbonate and caesium bicarbonate or combination thereof.

In another embodiment, the base used in step-iv) for formation Base salt of 6-carboxy-2-(3,5-dichlorophenyl)-benzoxazole of formula (I) is including but not limited to alkylamines selected from ethylamine, mono-, di- or triethanolamine, mono- and dipropanolamine, methyldiethanolamine, dipropanolamine, butyldiethanolamine, diethyl ethanolamine, isopropylamine, diisopropylamine, diisopropylethylamine, butylamine, t-butylamine, piperazine, piperidine, morpholine, cyclopentylamine, cyclohexylamine, dicyclohexylamine, aniline, benzylamine, diphenylamine, dibenzylamino, N-methylbenzylamine, naphthylethylamine, N-methylpiperazine, pyridine, phenetidine, lutidines, piperidine, 2-methyl piperidine, 5-dimethylaminopyridine and quinoline. These amine salts are increasing the solubility of formula (I) and it is very useful to purify the compound of formula (I).

In still another embodiment, the base salt of 6-carboxy-2-(3,5-dichlorophenyl) -benzoxazole of formula (I) is neutralised with acid such as hydrochloric acid, sulfuric acid, acetic acid and trifluoroacetic acid preferably hydrochloric acid to obtain 6-carboxy-2-(3,5-dichlorophenyl)-benzoxazole of formula (I) which is further converted into pharmaceutically acceptable salt of 6-carboxy-2-(3,5-dichlorophenyl) -benzoxazole of formula (I) wherein the neutralisation and salt formation steps are carried out without isolation of 6-carboxy-2-(3,5-dichlorophenyl)-benzoxazole of formula (I).

In still another embodiment, the ester hydrolysis, salt formation and pharmaceutically acceptable salt preparation steps are carried out without isolating intermediate compounds.

Still another embodiment, the novel Base salts of 6-carboxy-2-(3,5-dichlorophenyl)-benzoxazole of formula (I) disclosed herein are identified by several analytical parameters, alone or combination such as, but not limited to NMR, powder X-ray diffraction pattern (PXRD), differential scanning calorimetry and Infrared spectra.

One aspect of the present disclosure provides crystalline diethanolamine salt of 6-carboxy-2-(3,5-dichlorophenyl)-benzoxazole of formula (I). The diethanolamine salt of 6-carboxy-2-(3,5-dichlorophenyl)-benzoxazole of formula (I) has a PXRD pattern comprising peaks at 8.91, 16.7, 19.9 and 22.8±2θ. In another embodiment of the present disclosure, the diethanolamine salt of 6-carboxy-2-(3,5-dichlorophenyl) -benzoxazole of formula (I) has PXRD pattern at 8.91, 9.28, 11.34, 12.96, 13.82, 16.76, 17.76, 19.9, 22.79, 23.25, 27.55 and 29.00±2θ. Still one more aspect of the present disclosure the diethanolamine salt of 6-carboxy-2-(3,5-dichlorophenyl) -benzoxazole was characterised by PXRD pattern as shown following table-3.

TABLE 3
PXRD Results of Diethanolamine salt of 6-carboxy-2-(3,5-
dichlorophenyl)-benzoxazole

| Pos. [º2θ] | d-spacing [Å] | Rel. Int. [%] |
|---|---|---|
| 5.6467 | 15.65133 | 5.48 |
| 8.9170 | 9.91728 | 59.88 |
| 9.2856 | 9.52435 | 33.74 |
| 9.9363 | 8.90203 | 5.78 |
| 11.3472 | 7.79820 | 39.26 |
| 12.5183 | 7.07113 | 18.61 |
| 12.9608 | 6.83072 | 44.63 |
| 13.4080 | 6.60389 | 25.81 |
| 13.8237 | 6.40622 | 37.91 |
| 14.4670 | 6.12276 | 17.08 |
| 16.2798 | 5.44483 | 18.09 |
| 16.7087 | 5.30601 | 80.14 |
| 17.0531 | 5.19963 | 45.75 |
| 17.7666 | 4.99238 | 55.76 |
| 18.7656 | 4.72881 | 6.31 |
| 19.4884 | 4.55504 | 10.77 |
| 19.9770 | 4.44472 | 100.00 |
| 20.3188 | 4.37070 | 17.84 |
| 21.5894 | 4.11627 | 24.71 |
| 21.9879 | 4.04255 | 21.37 |
| 22.7934 | 3.90149 | 67.39 |
| 23.2581 | 3.82457 | 58.42 |
| 23.5827 | 3.77266 | 49.26 |
| 24.1479 | 3.68563 | 27.31 |
| 24.5348 | 3.62837 | 21.98 |
| 25.1032 | 3.54750 | 47.92 |
| 25.8280 | 3.44956 | 47.91 |
| 26.1659 | 3.40578 | 41.81 |
| 26.5265 | 3.36029 | 22.81 |
| 26.9442 | 3.30915 | 26.39 |
| 27.5555 | 3.23710 | 45.52 |
| 28.2138 | 3.16305 | 15.33 |
| 29.0279 | 3.07617 | 32.29 |
| 29.8493 | 2.99336 | 14.66 |
| 30.5705 | 2.92437 | 8.94 |
| 30.9548 | 2.88894 | 10.57 |
| 31.9430 | 2.80178 | 11.41 |
| 32.3502 | 2.76744 | 18.28 |
| 39.4076 | 2.28658 | 8.29 |
| 40.6057 | 2.22183 | 6.08 |

One aspect of the present disclosure provides novel pyridine salt of 6-carboxy -2-(3,5-dichlorophenyl)-benzoxazole. The pyridine salt of 6-carboxy-2-(3,5-dichlorophenyl)-benzoxazole has a PXRD pattern comprising peaks at 8.24, 9.99, 13.3, 17.33, 26.14 and 27.16±2θ. Still one more aspect of the present disclosure the pyridine salt of 6-carboxy-2-(3,5-dichlorophenyl)-benzoxazole was characterised by PXRD pattern as shown in following table-4.

TABLE 4
PXRD Results of pyridine salt of 6-carboxy-2-(3,5-
dichlorophenyl)-benzoxazole

| Pos. [º2θ] | d-spacing [Å] | Rel. Int. [%] |
|---|---|---|
| 8.2405 | 10.72976 | 24.53 |
| 9.9908 | 8.85366 | 38.61 |
| 11.0628 | 7.99801 | 21.57 |
| 11.7555 | 7.52822 | 8.55 |
| 13.3159 | 6.64934 | 39.68 |
| 14.4540 | 6.12824 | 16.80 |
| 15.6611 | 5.65850 | 11.07 |
| 16.1793 | 5.47843 | 13.76 |
| 16.6409 | 5.32748 | 41.46 |
| 17.3337 | 5.11610 | 64.89 |
| 17.7232 | 5.00452 | 8.36 |
| 18.9355 | 4.68675 | 35.71 |
| 19.5255 | 4.54646 | 36.05 |
| 20.3945 | 4.35465 | 31.06 |
| 22.2416 | 3.99702 | 16.48 |
| 23.5677 | 3.77503 | 38.28 |
| 24.5391 | 3.62775 | 21.74 |
| 26.1425 | 3.40878 | 86.32 |
| 27.1605 | 3.28328 | 100.00 |
| 28.5159 | 3.13023 | 16.45 |
| 30.4165 | 2.93883 | 7.61 |
| 32.0386 | 2.79364 | 7.41 |
| 33.7086 | 2.65896 | 7.58 |
| 35.0405 | 2.56089 | 3.22 |
| 35.7750 | 2.50998 | 4.27 |
| 36.6243 | 2.45370 | 6.64 |
| 38.2139 | 2.35521 | 3.64 |
| 44.1207 | 2.05264 | 1.25 |

One aspect of the present disclosure provides novel dicyclohexylamine salt of 6-carboxy-2-(3,5-dichlorophenyl)-benzoxazole. The dicyclohexylamine salt of 6-carboxy-2-(3,5-dichlorophenyl)-benzoxazole has a PXRD pattern comprising peaks at 8.00, 10.63, 15.68, 21.52 and 28.99±2θ. In another embodiment of the present disclosure, the dicyclohexylamine salt of 6-carboxy-2-(3,5-dichlorophenyl) -benzoxazole has PXRD pattern at 6.80, 7.17, 8.00, 10.63, 13.82, 15.68, 19.17, 20.63, 21.52, 22.32, 23.95 and 28.99±2θ. Still one more aspect of the present disclosure the dicyclohexylamine salt of 6-carboxy-2-(3,5-dichlorophenyl)-benzoxazole was characterised by PXRD pattern as shown in following table-5.

TABLE 5
PXRD Results of dicyclohexylamine salt of 6-carboxy-2-(3,5-
dichlorophenyl)-benzoxazole

| Pos. [º2θ] | d-spacing [Å] | Rel. Int. [%] |
|---|---|---|
| 6.8902 | 12.82918 | 15.36 |
| 7.1782 | 12.31523 | 14.89 |
| 8.0080 | 11.04084 | 76.13 |
| 10.0916 | 8.76538 | 8.94 |
| 10.6340 | 8.31952 | 42.47 |
| 11.0294 | 8.02214 | 8.23 |
| 13.8207 | 6.40759 | 10.52 |
| 14.3860 | 6.15707 | 8.36 |
| 14.6616 | 6.04194 | 10.55 |
| 15.8679 | 5.58522 | 30.28 |
| 16.2508 | 5.45447 | 18.05 |
| 18.6198 | 4.76551 | 9.40 |
| 19.1746 | 4.62886 | 25.10 |
| 20.1255 | 4.41224 | 20.22 |
| 20.6396 | 4.30348 | 25.75 |
| 21.5288 | 4.12771 | 100.00 |
| 22.3276 | 3.98182 | 12.17 |
| 22.7382 | 3.91082 | 6.52 |
| 23.3552 | 3.80889 | 8.05 |
| 23.9540 | 3.71502 | 22.76 |
| 24.2944 | 3.66373 | 15.17 |
| 25.7623 | 3.45821 | 7.09 |
| 26.3575 | 3.38145 | 7.97 |
| 26.7206 | 3.33632 | 5.43 |
| 27.4266 | 3.25202 | 6.19 |
| 27.8121 | 3.20782 | 7.64 |
| 28.3048 | 3.15309 | 5.03 |
| 28.9965 | 3.07943 | 36.32 |
| 30.3935 | 2.94099 | 3.10 |

Another aspect of the present disclosure provides novel t-butylamine salt of 6-carboxy-2-(3,5-dichlorophenyl)-benzoxazole. The t-butylamine salt of 6-carboxy-2-(3,5-dichlorophenyl)-benzoxazole has a PXRD pattern comprising peaks at 9.65, 14.52, 19.41 and 24.68±2θ. In another embodiment of the present disclosure , the t-butylamine salt of 6-carboxy-2-(3,5-dichlorophenyl)-benzoxazole has PXRD pattern at 9.65, 11.72, 14.52, 16.35, 19.41, 20.82, 22.78, 24.68, 25.26 and 34.31±0.2 °2θ. Still one more aspect of the present disclosure the t-butylamine salt of 6-carboxy-2-(3,5-dichlorophenyl)-benzoxazole was characterised by PXRD pattern as shown in following table-6.

TABLE 6
PXRD Results of t-butylamine salt of 6-carboxy-2-(3,5-
dichlorophenyl)-benzoxazole

| Pos. [º2θ] | d-spacing [Å] | Rel. Int. [%] |
|---|---|---|
| 9.6531 | 9.16261 | 10.13 |
| 11.7250 | 7.54776 | 19.62 |
| 13.7315 | 6.44901 | 12.92 |
| 14.5290 | 6.09678 | 43.38 |
| 16.3525 | 5.42080 | 19.74 |
| 17.7156 | 5.00666 | 9.00 |
| 18.8678 | 4.70343 | 12.39 |
| 19.4135 | 4.57242 | 100.00 |
| 20.8254 | 4.26552 | 31.81 |
| 21.3271 | 4.16629 | 7.42 |
| 21.9628 | 4.04712 | 4.12 |
| 22.7852 | 3.90287 | 45.73 |
| 23.1808 | 3.83716 | 17.39 |
| 23.6194 | 3.76688 | 14.20 |
| 24.6845 | 3.60671 | 53.91 |
| 25.2689 | 3.52461 | 33.09 |
| 26.8095 | 3.32546 | 7.06 |
| 27.8306 | 3.20573 | 6.72 |
| 28.6588 | 3.11495 | 20.56 |
| 29.3274 | 3.04544 | 13.81 |
| 32.1159 | 2.78709 | 4.37 |
| 32.6597 | 2.74192 | 2.10 |
| 33.2946 | 2.69107 | 2.36 |
| 33.7823 | 2.65333 | 2.77 |
| 34.3171 | 2.61320 | 18.09 |
| 35.2945 | 2.54304 | 5.79 |
| 38.2953 | 2.35039 | 2.14 |
| 39.4121 | 2.28633 | 3.45 |
| 40.6921 | 2.21731 | 5.03 |

Still one more embodiment of the present disclosure, crystalline Base salt of 6-carboxy-2-(3,5-dichlorophenyl)-benzoxazole of formula (II) can be converted in to other solid forms of 6-carboxy-2-(3,5-dichlorophenyl)-benzoxazole of formula (I) and the solid forms of 6-carboxy-2-(3,5-dichlorophenyl)-benzoxazole meglumine salt of Formula (IA)

Still one more embodiment, the pharmaceutically acceptable salt of 6-carboxy -2-(3,5-dichlorophenyl)-benzoxazole of formula (I) is meglumine salt of Formula-IA. The 6-carboxy-2-(3,5-dichlorophenyl)-benzoxazole meglumine salt of Formula-IA is prepared by adding meglumine to the reaction mass as a solid or it is dissolved in solvent and then added.

Still one more embodiment, the process for the preparation of 6-carboxy-2-(3,5-dichlorophenyl)-benzoxazole meglumine salt by adding 6-carboxy-2-(3,5-dichlorophenyl)-benzoxazole of formula (I) with suitable solvent selected from water, ethyl acetate, methyl acetate, toluene, hexane, heptane, acetonitrile, acetone, methyl isobutyl ketone, isopropyl ether, methyl tertiary butyl ether, dioxane, tetrahydrofuran, methanol, ethanol, isopropyl alcohol, isobutyl alcohol, butanol, isobutanol and pentanol or combination thereof, adding meglumine to the reaction mass, stirring the reaction mass at temperature in the range of 25° C. to 70° C. and filtering the stable polymorph of 6-carboxy-2-(3,5-dichlorophenyl)-benzoxazole meglumine of Formula-IA.

Still one more embodiment, the process for the preparation of meglumine salt of 6-carboxy-2-(3,5-dichlorophenyl)-benzoxazole of Formula-IA with/without isolating 6-carboxy-2-(3,5-dichlorophenyl)-benzoxazole of formula (I). If the 6-carboxy-2-(3,5-dichlorophenyl)-benzoxazole of formula (I) is isolated from the reaction mass, the PXRD of solid obtained from the reaction mass is matching with mixture of Form-4 and Form 6 as described in U.S. Pat. No. 9,770,441.

Still one more embodiment, the hydrate form of 6-carboxy-2-(3,5-dichlorophenyl)-benzoxazole meglumine of formula (IA) having characterised PXRD pattern comprising peaks at 3.85, 6.62, 7.63, 10.01, 11.44, 13.75, 16.63, 21.28, 22.95 and 24.43±2° 2 theta.

Still one more embodiment, the hydrate form of 6-carboxy-2-(3,5-dichlorophenyl)-benzoxazole meglumine salt of Formula-IA having a PXRD pattern comprising peaks at 3.85, 6.62, 7.63, 10.09, 11.44, 13.75, 16.63 and 22.65±0.2 °2θ and further comprising peaks as given in the table-7.

TABLE 7
PXRD Results of hydrate form of 6-carboxy-2-(3,5-
dichlorophenyl)-benzoxazole meglumine salt

| Pos. [º2θ] | d-spacing [Å] | Rel. Int. [%] |
|---|---|---|
| 3.8563 | 22.91319 | 33.85 |
| 6.6189 | 13.35449 | 47.94 |
| 7.6364 | 11.57719 | 81.22 |
| 10.0953 | 8.76225 | 100.00 |
| 11.4401 | 7.73505 | 39.51 |
| 13.2081 | 6.70337 | 24.82 |
| 13.7534 | 6.43880 | 72.77 |
| 15.2610 | 5.80593 | 17.52 |
| 16.6369 | 5.32876 | 37.68 |
| 19.0940 | 4.64822 | 24.07 |
| 19.8398 | 4.47514 | 18.80 |
| 20.2223 | 4.39135 | 38.80 |
| 21.2848 | 4.17449 | 22.50 |
| 22.9512 | 3.87501 | 97.95 |
| 24.4339 | 3.64313 | 38.15 |
| 25.1132 | 3.54611 | 21.90 |
| 25.9050 | 3.43949 | 19.58 |
| 27.0169 | 3.30040 | 14.41 |
| 27.6895 | 3.22174 | 7.70 |
| 28.5831 | 3.12302 | 6.49 |
| 29.1567 | 3.06287 | 9.24 |
| 30.0121 | 2.97750 | 5.48 |
| 30.8266 | 2.90066 | 7.06 |
| 31.5111 | 2.83920 | 11.99 |
| 32.8951 | 2.72284 | 12.79 |
| 33.5908 | 2.66802 | 13.11 |
| 34.2443 | 2.61858 | 3.71 |
| 35.2781 | 2.54418 | 2.55 |
| 36.8390 | 2.43989 | 7.00 |
| 37.3144 | 2.40989 | 2.66 |
| 38.3784 | 2.34549 | 2.35 |
| 39.2744 | 2.29403 | 4.50 |
| 40.4502 | 2.23002 | 5.52 |
| 40.8561 | 2.20879 | 11.45 |
| 42.6236 | 2.12121 | 3.50 |
| 44.6197 | 2.03084 | 23.57 |

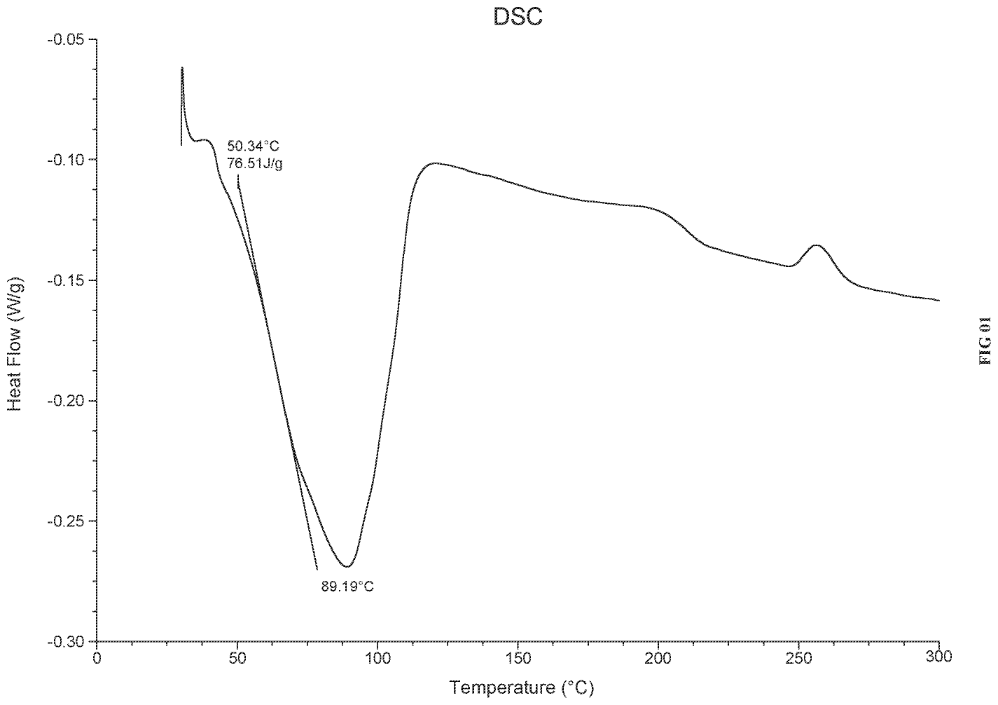

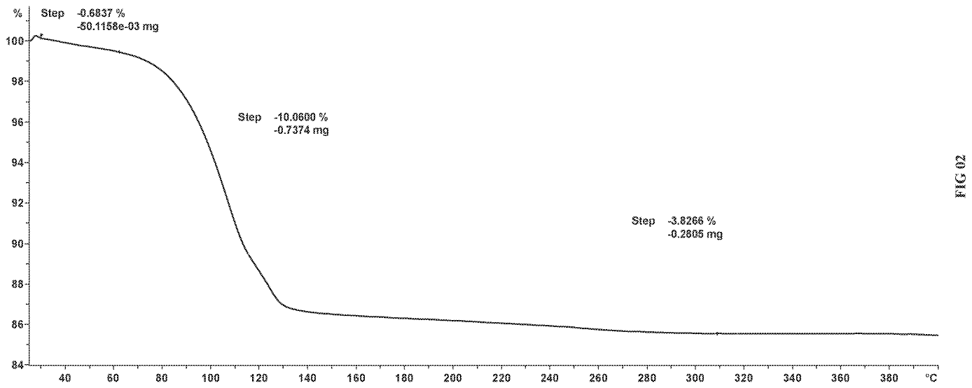

The hydrate form of 6-carboxy-2-(3,5-dichlorophenyl)-benzoxazole meglumine of Formula-IA according to the present disclosure having moisture content in the range of about 3.5% to about 9% preferably about 5 to 7%. The hydrate form of 6-carboxy-2-(3,5-dichlorophenyl)-benzoxazole meglumine salt of Formula-IA having moisture up to 15 to 20% but the only 5 to 7% water is present in crystal lattice and the excess water molecule is present in surface of the Tafamidis meglumine crystal. The present inventors are tried to remove the water present in the hydrate form of 6-carboxy-2-(3,5-dichlorophenyl)-benzoxazole meglumine salt of Formula-IA having moisture content about 6.64% but the water content is not considerably reduced even after drying at 55° C. temperature for 24 hours. The hydrate form of 6-carboxy-2-(3,5-dichlorophenyl)-benzoxazole meglumine salt of Formula-IA having moisture content about 6.64% and moisture content about 15% are resulting the same PXRD diffractogram. This confirms the water molecules up to moisture content about 6% is present in crystal lattice and the excess water is present in surface of the crystal. In an another embodiment, the present disclosure, the hydrate form of 6-carboxy-2-(3,5-dichlorophenyl)-benzoxazole meglumine having DSC as depicted in FIG. 1 and having TGA as depicted in FIG. 2.

In an embodiment of the present disclosure provides process for the preparation of hydrate form of 6-carboxy-2-(3,5-dichlorophenyl)-benzoxazole meglumine comprising
a. providing 6-carboxy-2-(3,5-dichlorophenyl)-benzoxazole in a solvent:
b. adding water to the reaction mass;
c. adding meglumine to the reaction mass;
d. optionally heating the reaction mass;
e. stirring the reaction mass; and
f. isolating hydrate form of 6-carboxy-2-(3,5-dichlorophenyl) -benzoxazole meglumine

wherein solvent in step a) is selected from ethyl acetate, methyl acetate, toluene, hexane, heptane, acetonitrile, acetone, methyl isobutyl ketone, isopropyl ether, methyl tertiary butyl ether, dioxane, tetrahydrofuran, methanol, ethanol, isopropyl alcohol, isobutyl alcohol, butanol, isobutanol and pentanol or combination thereof.

The starting material 4-(3,5-dichloro-benzoylamino)-3-hydroxy-benzoic acid of formula (V) of the present disclosure is prepared by conventional methods.

The present disclosure is provided by the examples below, which are provided by way of illustration only and should not be considered to limit the scope of this invention.

The date of powder X-ray diffraction pattern was measured with PANalytical X-ray Diffractometer equipped with CuKα radiations source=1.54184 Å° in a wide-angle. The instrument was operated in the continuous scan mode in step size 0.0167 per step and in the angular range of 5-50° (2θ).

The data of differential scanning calorimetry (DSC) are acquired by a TA Instruments Q20, with Thermal Advantage as instrument control software. Generally, 1-10 mg of sample is put into an aluminum crucible (unless otherwise specified, the aluminum crucible is covered). The temperature of sample was raised from room temperature to 350° C. with a heating rate of 5° C./min under the protection of dry nitrogen with a flow rate of 50 mL/min, while the TA software records the heat change of the sample during the heating process.

The data of thermogravimetric analysis (TGA) are acquired by a METTLER TOLEDO instrument with STARe software. Generally, 5-15 mg of sample is put into a Alumina 70 ul sample holder With segmented high resolution detection, the temperature of sample was raised from room temperature to 400° C. with a heating rate of 10° C./min under the protection of dry nitrogen with a flow rate of 60 mL/min, while the STARe software records the weight change of the sample during the heating process.

### EXAMPLE 1

### Preparation of 4-(3,5-dichloro-benzoylamino)-3-hydroxy-benzoic acid

To the stirred solution of 3,5-dichlorobenzoic acid in tetrahydrofuran, oxalyl chloride was added and stirred. To the reaction mass dimethylformamide was slowly added and stirred for 2-3 hrs. After completion of the reaction, the solvent from reaction mass was distilled completely. The obtained reaction mass was dissolved in tetrahydrofuran and 4-amino-3-hydroxy-benzoic acid was added at 25-35° C. and stirred for 3 hrs. The reaction mass was quenched with water followed by triethylamine. The obtained wet solid was slurred in aq. isopropanol solution. The obtained wet solid was slurred in dichloromethane, filtered, suck dried and dried under vacuum to obtain 4-(3,5-dichloro-benzoylamino)-3-hydroxy-benzoic acid.

### Yield: 88%.

### EXAMPLE 2

### 2-(3,5-Dichlorophenyl)-benzoxazole-6-carboxylic acid methyl ester of Formula (VIII)

To the stirred solution of 4-(3,5-dichloro-benzoylamino)-3-hydroxy-benzoic acid in tetrahydrofuran at 30±5° C. thionyl chloride was slowly added followed by methanol. The reaction was heated to 50° C. and stirred. After completion of the reaction, the reaction mass was distilled, the residue obtained was slurred in methanol, filtered and dried to obtain 4-(3,5-dichloro-benzoylamino)-3-hydroxy-benzoic acid methyl ester of formula (VII).

To the stirred solution of 4-(3,5-dichloro-benzoylamino)-3-hydroxy-benzoic acid methyl ester of formula (VII) in toluene at 30° C. p-toluene sulfonic acid was charged, the reaction mass was heated to azeotropic reflux temperature under stirring. After completion of the reaction, the solvent was distilled and the obtained solid was slurred in methanol and filtered and dried to obtain 2-(3,5-dichlorophenyl)-benzoxazole-6-carboxylic acid methyl ester of Formula (VIII).

### EXAMPLE 3

### Process for the preparation of 6-carboxy-2-(3,5-dichlorophenyl) -benzoxazole meglumine (Form M)

To the stirred solution of aq. lithium hydroxide (52.5 g in 500 L water), 2.2 Kg of tetrahydrofuran and 100 g of 2-(3,5-dichlorophenyl)-benzoxazole-6-carboxylic acid methyl ester of Formula (VIII) was added. The reaction mass was stirred at 30±5° C. for 15 hrs. After completion of the reaction, the pH of the reaction mass was adjusted to acidic at 30±5° C. with dil. hydrochloric acid and separated the layers, Aq. layer back extracted with tetrahydrofuran, combined organic layers washed with brine solution and separated the layers, charcoal treatment given to organic layer and filtered. To the filtrate 48 g of meglumine was added at 30±5° C. and stirred at 30±5° C. for 6 hr. After completion of the reaction, obtained solid was filtered, washed with tetrahydrofuran and dried to obtain Form M of 6-carboxy-2-(3,5-dichlorophenyl)-benzoxazole meglumine.

### EXAMPLE 4

### Preparation of 6-carboxy-2-(3,5-dichlorophenyl)-benzoxazole Diethanolamine salt

To a solution of toluene in 4-(3,5-dichloro-benzoylamino)-3-hydroxy-benzoic acid of formula (V), p-toluenesulfonic acid was added and stirred. The reaction was heated to 110-140° C. and stirred for 40 hours. After completion of the reaction, the mass was stirred with aq. tetrahydrofuran and filtered. A solution of 6-carboxy-2-(3,5-dichlorophenyl)-benzoxazole was stirred at 25-35° C. The reaction mass was warmed and cooled to room temperature. To the reaction mass diethanolamine was added and stirred for 4 hours. The obtained solid was filtered and washed with tetrahydrofuran to obtain 6-carboxy-2-(3,5-dichlorophenyl)-benzoxazole diethanolamine salt.

### EXAMPLE 5

### Preparation of 6-carboxy-2-(3,5-dichlorophenyl)-benzoxazole pyridine salt

To a solution of toluene in 4-(3,5-dichloro-benzoylamino)-3-hydroxy-benzoic acid of formula (V), p-toluenesulfonic acid was added and stirred. The reaction was heated to 110-140° C. and stirred for 40 hours. After completion of the reaction, the mass was stirred with aq. tetrahydrofuran and filtered. A solution of 6-carboxy-2-(3,5-dichlorophenyl)-benzoxazole was stirred at 25-35° C. The reaction mass was warmed and cooled to room temperature. To the reaction mass pyridine was added and stirred for 4 hours. The obtained solid was filtered and washed with tetrahydrofuran to obtain 6-carboxy-2-(3,5-dichlorophenyl)-benzoxazole pyridine salt.

### EXAMPLE 6

### Preparation of 6-carboxy-2-(3,5-dichlorophenyl)-benzoxazole dicyclohexylamine salt

To a solution of toluene in 4-(3,5-dichloro-benzoylamino)-3-hydroxy-benzoic acid of formula (V), p-toluenesulfonic acid was added and stirred. The reaction was heated to 110-140° C. and stirred for 40 hours. After completion of the reaction, the mass was stirred with aq. tetrahydrofuran and filtered. A solution of 6-carboxy-2-(3,5-dichlorophenyl)-benzoxazole was stirred at 25-35° C. The reaction mass was warmed and cooled to room temperature. To the reaction mass dicyclohexylamine was added and stirred for 4 hours. The obtained solid was filtered and washed with tetrahydrofuran to obtain 6-carboxy-2-(3,5-dichlorophenyl)-benzoxazole dicyclohexylamine salt

### EXAMPLE 7

### Preparation of 6-carboxy-2-(3,5-dichlorophenyl)-benzoxazole t-butylamine salt

To a solution of toluene in 4-(3,5-dichloro-benzoylamino)-3-hydroxy-benzoic acid of formula (V), p-toluenesulfonic acid was added and stirred. The reaction mass was heated to 110-140° C. and stirred for 40 hours. After completion of the reaction, the mass was stirred with aq. tetrahydrofuran and filtered. A solution of 6-carboxy-2-(3,5-dichlorophenyl)-benzoxazole was stirred at 25-35° C. To the reaction mass t-butylamine was added and stirred for 4 hours. The obtained solid was filtered and washed with tetrahydrofuran to obtain 6-carboxy-2-(3,5-dichlorophenyl)-benzoxazole t-butylamine salt.

### EXAMPLE 8

### General process for the preparation of 6-carboxy-2-(3,5-dichlorophenyl)-benzoxazole meglumine from 6-carboxy-2-(3,5-dichlorophenyl) -benzoxazole Base salt

To a solution of toluene in 4-(3,5-dichloro-benzoylamino)-3-hydroxy-benzoic acid of formula (V), p-toluenesulfonic acid was added and stirred. The reaction was heated to 110-140° C. and stirred for 40 hours. After completion of the reaction the reaction, the mass was stirred with aqueous tetrahydrofuran and filtered. To the filtrate containing 6-carboxy-2-(3,5-dichlorophenyl)-benzoxazole, corresponding Base was added, stirred for 4 hours and filtered to obtain Base salt of 6-carboxy-2-(3,5-dichlorophenyl)-benzoxazole. To the wet solid aqueous tetrahydrofuran was added and the pH was adjusted with hydrochloric acid. To the reaction mass water was added and layers were separated. To the organic layer meglumine was added, heated to 50 to 60° C. and stirred for 24 hours. The obtained solid is filtered and washed with acetone and dried to obtain stable crystalline Form-M of 6-carboxy-2-(3,5-dichlorophenyl)-benzoxazole meglumine of Formula-IA.

### EXAMPLE 9

To a 20 g of 4-(3,5-dichloro-benzoylamino)-3-hydroxy-benzoic acid, 200 mL methane sulfonic acid was added and stirred. The reaction mass was heated to 80-120° C. and stirred for 24 hours. The reaction mass was cooled to room temperature. To the solution 1000 mL n-butanol was added lot wise (200 mL×5) and stirred for 2 hr. The obtained solid was filtered and dried.

### PXRD: Form L

### EXAMPLE 10

To a 20 g of 4-(3,5-dichloro-benzoylamino)-3-hydroxy-benzoic acid, 200 mL methane sulfonic acid was added and stirred. The reaction mass was heated to 80-120° C. and stirred for 24 hours. The reaction mass was cooled to room temperature. To the solution 800 mL of n-butanol was added and stirred for 1 hr. The obtained solid was filtered and dried.

### PXRD: Form P:

### EXAMPLE 11

### Process for the Preparation of Form-4

To a 2 g of 4-(3,5-dichloro-benzoylamino)-3-hydroxy-benzoic acid, 20 mL methane sulfonic acid was added and stirred. The reaction mass heated to 80-120° C. and stirred for 24 hours. The reaction mass was cooled to room temperature. To the solution 80 mL of isopropanol was added and stirred for 1 hr. The obtained solid was filtered and dried.

### PXRD: Form-4

### EXAMPLE 12

### Process for the Preparation of Form-4

To a 2 g of 4-(3,5-dichloro-benzoylamino)-3-hydroxy-benzoic acid, 20 mL methane sulfonic acid was added and stirred. The reaction mass was heated to 80-120° C. and stirred for 24 hours. The reaction mass was cooled to room temperature. To the solution 80 mL methanol was added and stirred for 1 hr. The obtained solid was filtered and dried.

### PXRD: Form-4

### EXAMPLE 13

### Process for the Preparation of Form-4

To a 2 g of 4-(3,5-dichloro-benzoylamino)-3-hydroxy-benzoic acid, 20 mL methane sulfonic acid was added and stirred. The reaction mass heated to 80-120° C. and stirred for 24 hours. The reaction mass was cooled to room temperature. To the solution 80 mL acetone was added and stirred for 1 hr. The obtained solid was filtered and dried.

### PXRD: Form-4

### EXAMPLE 14

### Process for the Preparation of Form-4

To a 3 g of 4-(3,5-dichloro-benzoylamino)-3-hydroxy-benzoic acid, 30 mL methane sulfonic acid was added and stirred. The reaction mass heated to 80-120° C. and stirred for 24 hours. The reaction mass was cooled to room temperature. To the solution 120 mL dioxane was added and stirred for 1 hr. The obtained solid was filtered and dried.

### PXRD: Form-4

### EXAMPLE 15

### Process for the Preparation of Form-4

To a 10 g of 4-(3,5-dichloro-benzoylamino)-3-hydroxy-benzoic acid, 100 mL methane sulfonic acid was added and stirred. The reaction mass heated to 80-120° C. and stirred for 24 hours. The reaction mass was cooled to room temperature. To the solution 400 mL of methyl tertiarybutyl ether was added and stirred for 1 hr. The obtained solid was filtered and dried.

### PXRD: Form-4

### EXAMPLE 16

### Process for the Preparation of Form-4

To a 10 g of 4-(3,5-dichloro-benzoylamino)-3-hydroxy-benzoic acid, 100 mL methane sulfonic acid was added and stirred. The reaction mass heated to 80-120° C. and stirred for 24 hours. The reaction mass was cooled to room temperature. To the solution 400 mL of diisopropylether was added and stirred for 1 hr. The obtained solid was filtered and dried.

### PXRD: Form-4

### EXAMPLE 17

### Process for the Preparation of Form-4

To a 2 g of 4-(3,5-dichloro-benzoylamino)-3-hydroxy-benzoic acid, 20 mL methane sulfonic acid was added and stirred. The reaction mass heated to 80-120° C. and stirred for 24 hours. The reaction mass was cooled to room temperature. To the solution 80 mL of acetonitrile was added and stirred for 1 hr. The obtained solid was filtered and dried.

### PXRD: Form-4

### EXAMPLE 18

### Process for the preparation of 6-carboxy-2-(3,5-dichlorophenyl) -benzoxazole meglumine

To a 60 mL of acetone 2 g of 6-carboxy-2-(3,5-dichlorophenyl)-benzoxazole Form 4 was added. The reaction mass was heated to 55-65° C., 1.28 g of meglumine was added to the reaction mass and stirred for 2 hrs. The reaction mass was cooled to 25-35° C. and the obtained solid was washed with acetone, filtered and dried to obtain stable crystalline Form-M of 6-carboxy-2-(3,5-dichlorophenyl)-benzoxazole meglumine of Formula-IA.

Dry weight 3.2 g

### EXAMPLE 19

### Process for the preparation of 6-carboxy-2-(3,5-dichlorophenyl) -benzoxazole meglumine of Formula-IA

To a 60 mL of acetone 2 g of 6-carboxy-2-(3,5-dichlorophenyl)-benzoxazole Form L or Form P was added. The reaction mass was heated to 55-65° C., 1.28 g of meglumine was added to the reaction mass and stirred for 2 hrs. The reaction mass was cooled to 25-35° C. and the obtained solid was washed with acetone, filtered and dried to obtain stable crystalline Form-M of 6-carboxy-2-(3,5-dichlorophenyl)-benzoxazole meglumine of Formula-IA. Dry weight 3.04 g

### EXAMPLE 20

### Process for the preparation of Hydrate form of 6-carboxy-2-(3,5-dichlorophenyl)-benzoxazole meglumine of Formula-IA

To the stirred solution of aq. lithium hydroxide (52.5 g in 500 L water), 2.2 Kg of tetrahydrofuran and 100 g of 2-(3,5-dichlorophenyl)-benzoxazole-6-carboxylic acid methyl ester of Formula (VIII) was added. The reaction mass was stirred at 30±5° C. for 15 hrs. After completion of the reaction, the reaction mass pH was adjusted to acidic at 30±5° C. with dil. hydrochloric acid. The reaction mass was treated with carbon, filtered and washed the bed with tetrahydrofuran. To the filtrate 48 g of meglumine was added at 30±5° C. and stirred at 30±5° C. for 6 hr. After completion of the reaction, the obtained solid was filtered, washed with tetrahydrofuran and dried to obtain Hydrate of 6-carboxy-2-(3,5-dichlorophenyl)-benzoxazole meglumine.

### EXAMPLE 21

### Process for the preparation of 6-carboxy-2-(3,5-dichlorophenyl) -benzoxazole meglumine

To the stirred solution of aq. lithium hydroxide (52.5 g in 500 L water), 2.2 Kg of tetrahydrofuran and 100 g of 2-(3,5-dichlorophenyl)-benzoxazole-6-carboxylic acid methyl ester of Formula (VIII) was added. The reaction mass was stirred at 30±5° C. for 15 hrs. After completion of the reaction, the pH of the reaction mass was adjusted to acidic at 30±5° C. with dil. hydrochloric acid and separated the layers, Aq. layer back extracted with tetrahydrofuran, combined organic layers washed with brine solution and separated the layers, charcoal treatment given to organic layer and filtered. To the filtrate, water was added stirred for 30 minutes, 48 g of meglumine was added at 30±5° C. and stirred at 30±5° C. for 6 hr. After completion of the reaction, obtained solid was filtered, washed with tetrahydrofuran and dried to obtain hydrate form of 6-carboxy-2-(3,5-dichlorophenyl)-benzoxazole meglumine.

1-4. (canceled)

5. A process for the preparation of Form-4 of 6-carboxy-2-(3,5-dichlorophenyl)-benzoxazole of formula (I) comprising:
a) providing compound of formula (V); 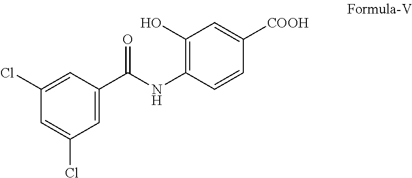 [SMILES: O=C(O)c1ccc(NC(=O)c2cc(Cl)cc(Cl)c2)c(O)c1]
b) adding acid to the reaction mass;
c) heating the reaction mass optionally in presence of solvent; and
d) adding suitable solvent to precipitate form-4 of compound of formula (I).
wherein the acid is selected from methane-sulfonic acid, ethanesulfonic acid, phenylmethanesulfonic acid, camphor-l0-sulfonic acid, naphthalene-1-sulfonic acid, naphthalene-2-sulfonic acid, naphthalene-1,5-disulfonic acid, sulfuric acid, fumaric acid, phosphoric acid and polyphosphoric acid or a combination thereof and suitable solvent in step d) is selected from methanol, ethanol, isopropanol, butanol, acetone, methyl ethyl ketone, methyl isobutyl ketone ethyl acetate, acetonitrile, diisopropylether, methyl tertiarybutyl ether, dioxane, toluene, anisole, hexane, cyclohexane and heptane or combination thereof.

6. The process according to the claim 5 wherein the acid used for cyclisation is methanesulfonic acid and the cyclisation reaction is carried out without solvent.

7. The process for the preparation of Form-4 according to the claim 5, wherein the Form-4 of 6-carboxy-2-(3,5-dichlorophenyl)-benzoxazole formula (I) is converted in to other solid forms of 6-carboxy-2-(3,5-dichlorophenyl)-benzoxazole formula (I) or solid forms of 6-carboxy-2-(3,5-dichlorophenyl)-benzoxazole meglumine salt of Formula (IA).

8. Base salt of 6-carboxy-2-(3,5-dichlorophenyl)-benzoxazole of formula (II) 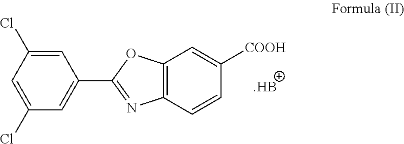
wherein the HB^{⊕} is basic addition salt.

9. The base salt of 6-carboxy-2-(3,5-dichlorophenyl) -benzoxazole of formula (II) according to the claim 8, wherein the base salt is selected from calcium, magnesium, ethanolamine, diethanolamine, pyridine, dicyclohexylamine, phenylethylamine, isopropylamine, diisopropylethylamine, triethylamine and t-butylamine.

10. The base salt of 6-carboxy-2-(3,5-dichlorophenyl)-benzoxazole of formula (II) according claim 8 wherein
a) crystalline diethanolamine salt of 6-carboxy-2-(3,5-dichlorophenyl)-benzoxazole has a PXRD pattern comprising peaks at 8.91, 16.7, 19.9 and 22.8±0.2° 2θ;
b) crystalline pyridine salt of 6-carboxy-2-(3,5-dichlorophenyl)-benzoxazole has a PXRD pattern comprising peaks at 8.24, 9.99, 13.3, 17.33, 26.14 and 27.16±0.2° 2θ;
c) crystalline Dicyclohexylamine salt of 6-carboxy-2-(3,5-dichlorophenyl) -benzoxazole has a PXRD pattern comprising peaks at 8.00, 10.63, 15.68, 21.52 and 28.99±0.2° 2θ; and
d) crystalline t-Butylamine salt of 6-carboxy-2-(3,5-dichlorophenyl)-benzoxazole of formula (I) has a PXRD pattern comprising peaks at 9.65, 14.52, 19.41 and 24.68±2θ.

11. A process for preparation of 6-carboxy-2-(3,5-dichlorophenyl)-benzoxazole of formula (I) and its pharmaceutically acceptable salt thereof comprising:
i. esterifying 4-(3,5-dichloro-benzoylamino)-3-hydroxy-benzoic acid of formula (V) 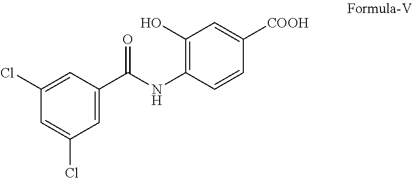 [SMILES: O=C(O)c1ccc(NC(=O)c2cc(Cl)cc(Cl)c2)c(O)c1]
to obtain 4-(3,5-dichloro-benzoylamino)-3-hydroxy-benzoic acid alkyl ester of formula (VII); 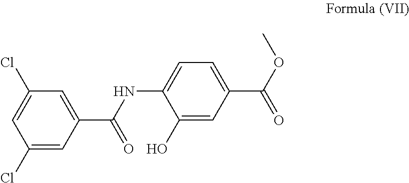 [SMILES: COC(=O)c1ccc(NC(=O)c2cc(Cl)cc(Cl)c2)c(O)c1]
ii. cyclizing 4-(3,5-dichloro-benzoylamino)-3-hydroxy-benzoic acid alkyl ester to obtain 2-(3,5-dichlorophenyl)-benzoxazole-6-carboxylic acid methyl ester of Formula (VIII); 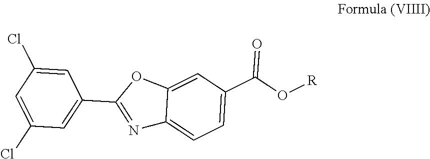
iii. converting 2-(3,5-dichlorophenyl)-benzoxazole-6-carboxylic acid alkyl ester of Formula (VIII) to 6-carboxy-2-(3,5-dichlorophenyl)-benzoxazole of formula (I) to its Base salt of formula (II); and 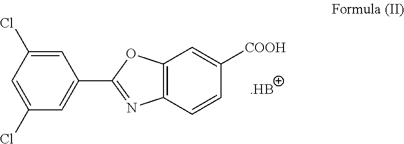
iv. converting 6-carboxy-2-(3,5-dichlorophenyl)-benzoxazole Base salt of formula (II) to 6-carboxy-2-(3,5-dichlorophenyl)-benzoxazole of formula (I) and its pharmaceutically acceptable salt.

12. The process according to the claim 11, wherein the esterification is carried out in presence of activating agent and alkanol wherein the activating agent is selected from thionyl chloride or oxalyl chloride and the alkanol is selected from methanol, ethanol, propanol, isopropanol, butanol, isobutanol, t-butanol, 1-pentanol and 2-pentanol.

13. The process according to the claim 11, wherein the cyclization of step ii) is carried out in presence of acid selected from p-toluenesulphonic acid, methane-sulfonic acid, ethanesulfonic acid, phenylmethanesulfonic acid, camphor-10-sulfonic acid, naphthalene-1-sulfonic acid, naphthalene-2-sulfonic acid, naphthalene-1,5-disulfonic acid and sulfuric acid.

14. The process according to the claim 11, wherein the pharmaceutically acceptable salts of 6-carboxy-2-(3,5-dichlorophenyl)-benzoxazole of formula (I) prepared by without isolation of 6-carboxy-2-(3,5-dichlorophenyl)-benzoxazole of formula (I).

15. A Hydrate form of 6-carboxy-2-(3,5-dichlorophenyl)-benzoxazole meglumine having water content about 3% to about 20%.

16. The Hydrate form of 6-carboxy-2-(3,5-dichlorophenyl) -benzoxazole meglumine according to the claim 15, wherein said hydrate having PXRD pattern comprising peaks at 6.62, 7.63, 10.09, 13.75, and 22.65±0.2 °2θ.

17. [Currently Amended] The Hydrate form of 6-carboxy-2-(3,5-dichlorophenyl) -benzoxazole meglumine according to the claim 15, wherein PXRD pattern further comprising peaks at 3.85, 11.44, 7.63, 16.63, 21.28 and 24.43±0.2 °2θ.

18. A process for the preparation of hydrate form of 6-carboxy -2-(3,5-dichlorophenyl)-benzoxazole meglumine according to claim 15 comprising:
a. providing 6-carboxy-2-(3,5-dichlorophenyl)-benzoxazole in a solvent:
b. adding water to the reaction mass;
c. adding meglumine to the reaction mass;
d. optionally heating the reaction mass;
e. stirring the reaction mass; and
f. isolating hydrate form of 6-carboxy-2-(3,5-dichlorophenyl)-benzoxazole meglumine
wherein solvent in step a) is selected from ethyl acetate, methyl acetate, toluene, hexane, heptane, acetonitrile, acetone, methyl isobutyl ketone, isopropyl ether, methyl tertiary butyl ether, dioxane, tetrahydrofuran, methanol, ethanol, isopropyl alcohol, isobutyl alcohol, butanol, isobutanol and pentanol or combination thereof.

19. A Pharmaceutical composition comprising the crystalline Hydrate form of claim 15 in therapeutically acceptable amount in admixture with at least one pharmaceutically acceptable excipient.

20. A method of treating transthyretin amyloid disease in mammal, the method comprising administering to the mammal a therapeutically effective amount of the crystalline hydrate form of claim 15.

21. The process according to the claim 5, wherein the reaction is carried out at temperature in the range of 80° C. to 120° C.
